(19) United States
(12) Patent Application Publication
(10) Pub. No.: US 2026/0120812 A1
(43) Pub. Date: Apr. 30, 2026

(54) OPTIMIZATION OF QUANTUM-ACTIVE DEFECTS AND SPINS IN CHEMICAL SYSTEMS USING MACHINE LEARNING

(71) Applicant: Massachusetts Institute of Technology, Cambridge, MA (US)

(72) Inventors: Dane William deQuilettes, Somerville, MA (US); Eden C. Price, Boston, MA (US); Swaroop Vattam, Westford, MA (US); Danielle Braje, Winchester, MA (US); Justin Lee Mallek, Burlington, MA (US); Jennifer May Schloss, Cambridge, MA (US); Linh M. Pham, Arlington, MA (US)

(73) Assignee: Massachusetts Institute of Technology, Cambridge, MA (US)

(21) Appl. No.: 19/157,859

(22) PCT Filed: Feb. 22, 2024
(86) PCT No.: PCT/US2024/016950

§ 371 Date: Aug. 19, 2025

Related U.S. Application Data
(60) Provisional application No. 63/486,459, filed on Feb. 22, 2023.

(51) Int. Cl. G16C 10/00; G16C 20/70

(52) U.S. Cl. CPC G16C 10/00; G16C 20/70

(57) ABSTRACT

Systems and methods for the manufacture of improved quantum materials are provided. The techniques include generating, using a machine learning model, a regression model of a figure of merit describing the quantum material, the regression model being determined based at least in part on the characterized one or more quantum properties of individual samples of the quantum material and associated fabrication parameters. The techniques also include determining improved fabrication parameters using the regression model of the figure of merit and fabricating a new sample of the quantum material using the improved fabrication parameters.

### CROSS REFERENCE TO RELATED APPLICATIONS

This application claims the benefit under 35 U.S.C. § 119(e) to U.S. Provisional Application No. 63/486,459, filed Feb. 22, 2023, titled “OPTIMIZATION OF QUANTUM-ACTIVE DEFECTS AND SPINS IN CHEMICAL SYSTEMS USING MACHINE LEARNING,” which is incorporated herein by reference in its entirety.

### FEDERALLY SPONSORED RESEARCH

This invention was made with government support under FA8702-15-D-0001 awarded by the U.S. Air Force. The government has certain rights in the invention.

### BACKGROUND

Quantum materials are, broadly, materials that have properties that cannot be easily described by classical physics. Rather, quantum materials exhibit properties resulting from, for example, electronic or other interactions at the atomic and subatomic scale. Quantum properties exhibited by quantum materials include quantum tunneling, entanglement, interference, and topological effects, among other properties.

### SUMMARY

Some embodiments are directed to a method of manufacturing a quantum material, the method comprising: fabricating a first sample of the quantum material using a first set of fabrication parameters; characterizing one or more quantum properties of the first sample; generating, using a machine learning model, a regression model of a figure of merit describing the quantum material, the regression model being determined based at least in part on the characterized one or more quantum properties of the first sample and the first set of fabrication parameters; determining improved fabrication parameters based at least in part on the regression model of the figure of merit; and fabricating a second sample of the quantum material using one or more of the improved fabrication parameters.

In some embodiments, fabricating the first sample and fabricating the second sample comprises fabricating diamond samples, the diamond samples including one or more color centers.

In some embodiments, fabricating the diamond samples including one or more color centers comprises fabricating diamond samples including one or more nitrogen-vacancy centers.

In some embodiments, generating the regression model comprises performing gradient boost regression, random forest regression, and/or stacking regression.

In some embodiments, determining the improved fabrication parameters comprises calculating SHAP values associated with fabrication parameters of the first set of fabrication parameters and predicted effects of the fabrication parameters on the figure of merit.

In some embodiments, determining the improved fabrication parameters comprises determining one of the first set of fabrication parameters having a largest impact on the figure of merit based at least in part on the calculated SHAP values.

In some embodiments, determining the improved fabrication parameters comprises determining the improved fabrication parameters using Bayesian optimization.

In some embodiments, using Bayesian optimization comprises determining local minima and/or maxima of the figure of merit as a function of one fabrication parameter of the first set of fabrication parameters to determine one of the improved fabrication parameters.

In some embodiments, generating the regression model based at least in part on the characterized one or more quantum properties of the first sample comprises generating the regression model based at least in part on one or more of measured T2* dephasing times, contrast, and/or fluorescence.

In some embodiments, generating the regression model based at least in part on the first set of fabrication parameters comprises generating the regression model based at least in part on one or more of seed miscut angles, growth time, seed depth, total irradiation dose, methane flow rate, nitrogen flow rate, pump down times, base pressure, and/or rate of rise.

Some embodiments are directed to a system, comprising: one or more devices configured to fabricate a first sample and a second sample of a quantum material; at least one computer processor; and at least one non-transitory computer readable medium storing instructions that, when executed by the at least one computer processor, cause the at least one computer processor to perform a method of improving manufacture of a quantum material, the method comprising: generating, using a machine learning model, a regression model of a figure of merit describing the quantum material, the regression model being determined based at least in part on characterized one or more quantum properties of the first sample and a first set of fabrication parameters used by the one or more devices to fabricate the first sample; and determining improved fabrication parameters based at least in part on the regression model of the figure of merit, the determined improved fabrication parameters to be used by the one or more devices to fabricate the second sample.

In some embodiments, the one or more devices are configured to fabricate diamond samples including one or more color centers.

In some embodiments, the one or more devices are configured to fabricate diamond samples including one or more nitrogen-vacancy centers.

In some embodiments, generating the regression model comprises performing gradient boost regression, random forest regression, and/or stacking regression.

In some embodiments, determining the improved fabrication parameters comprises calculating SHAP values associated with fabrication parameters of the first set of fabrication parameters and predicted effects of the fabrication parameters on the figure of merit.

In some embodiments, determining the improved fabrication parameters comprises determining one of the first set of fabrication parameters having a largest impact on the figure of merit based at least in part on the calculated SHAP values.

In some embodiments, determining the improved fabrication parameters comprises determining the improved fabrication parameters using Bayesian optimization.

In some embodiments, using Bayesian optimization comprises determining local minima and/or maxima of the figure of merit as a function of one fabrication parameter of the first set of fabrication parameters to determine one of the improved fabrication parameters.

In some embodiments, generating the regression model based at least in part on the characterized one or more quantum properties of the first sample comprises generating the regression model based at least in part on one or more of measured T2* dephasing times, contrast, and/or fluorescence.

In some embodiments, generating the regression model based at least in part on the first set of fabrication parameters comprises generating the regression model based at least in part on one or more of seed miscut angles, growth time, seed depth, total irradiation dose, methane flow rate, nitrogen flow rate, pump down times, base pressure, and/or rate of rise.

Some embodiments are directed to a quantum material fabricated using the techniques described herein.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1A is a schematic diagram of a diamond crystal structure including a nitrogen-vacancy center, in accordance with some embodiments of the technology described herein.

FIG. 1B is a schematic diagram of an energy level structure of a nitrogen-vacancy diamond system, in accordance with some embodiments of the technology described herein.

FIG. 2 is a schematic diagram of a manufacturing pipeline 200 configured to manufacture improved quantum materials, in accordance with some embodiments of the technology described herein.

FIG. 3 is a plot of emission spectra of a nitrogen-doped diamond as a function of applied laser power, in accordance with some embodiments of the technology described herein.

FIG. 4 is a plot of root-mean-square error (RMSE) of different machine learning models used to build regression models, in accordance with some embodiments of the technology described herein.

FIG. 5 is a plot of SHAP values calculated for different fabrication parameters, in accordance with some embodiments of the technology described herein.

FIG. 6A is a density contour plot of predicted figures of merit as a function of total dose, in accordance with some embodiments of the technology described herein.

FIG. 6B is a density contour plot of predicted figures of merit as a function of miscut angle, κ, in accordance with some embodiments of the technology described herein.

FIG. 7 is a correlation plot of machine-learning-predicted figure of merit values in comparison to measured figure of merit values, in accordance with some embodiments of the technology described herein.

FIG. 8 is a flowchart describing a process 800 of manufacturing a quantum material, in accordance with some embodiments of the technology described herein.

FIG. 9 is a schematic diagram of an illustrative implementation of a computer system that may be used in connection with some embodiments of the technology described herein.

### DETAILED DESCRIPTION

Described herein are techniques for manufacturing quantum materials with improved quantum material properties. The techniques include building, using a machine learning model, a regression model describing relationships between fabrication parameters associated with fabrication of the quantum material and measured properties of the quantum material. The regression model is then used to generate new combinations of fabrication parameters predicted to yield quantum materials having improved quantum properties.

As described herein, quantum materials are materials exhibiting properties that are not classical in nature. Quantum materials may be defined by the composition of material used (e.g., topological insulators, organic molecules, transition metal dichalcogenides, superconducting materials, etc.), by a unique geometry of the material (e.g., one- or two-dimensional materials), and/or by integration of the material into a quantum device (e.g., superconducting qubits, magnetometers, and/or other devices). Quantum materials have a broad array of applications, including but not limited to magnetic navigation, nanoscale nuclear magnetic resonance (NMR) spectroscopy, single molecular detection, telecommunications, computing, quantum sensing, and/or high resolution biosensing. However, the properties of quantum materials can be highly sensitive to parameters governing the fabrication of the quantum materials, and typically there are a large number of fabrication parameters that may affect the final properties of the quantum material.

The process of developing new and/or improved quantum materials conventionally requires manually fabricating samples of a quantum material using various fabrication parameters, characterizing the fabricated samples, and analyzing the resulting quantum properties. This is, however, a time-consuming and resource-intensive task, taking months or years and sufficient funding and staffing. Additionally, the relationships between fabrication parameters and/or resulting material properties are often multivariate in nature such that it can be difficult, if impossible, to ensure that all fabrication parameters have been globally optimized.

The inventors have recognized and appreciated that machine learning techniques can describe complex, nonlinear relationships, like those between fabrication parameters and quantum material performance, and may be used to determine improved fabrication parameters for quantum materials. The inventors have further recognized and appreciated that such machine learning optimization techniques may predict improved fabrication parameters at an accelerated pace relative to manual fabrication optimization and that, additionally, machine learning optimization techniques may predict combinations of fabrication parameters that would be unlikely to be discovered manually.

Accordingly, the inventors have developed techniques for improving properties of quantum materials by using machine learning to predict optimized material fabrication parameters. The techniques describe herein are broadly applicable to the fabrication of quantum materials including, but not limited to, chemical systems that can host quantum-active defects and spins (e.g., diamond, silicon carbide, transition metal dichalcogenides, yttrium iron garnet (YIG), organic molecules, metal centered inorganic complexes, etc.). In some embodiments, the techniques include generating a regression model describing a figure of merit associated with the quantum material. For example, the figure of merit may be dependent on one or more quantum properties of the quantum material, and improving (e.g., maximizing and/or minimizing) the figure of merit may correlate with improving the quantum properties of the quantum material for one or more particular applications. The regression model may be generated using supervised learning techniques including learning the regression model using pairs of calculated figures of merit (e.g., calculated based on one or more measured quantum properties of characterized samples of the quantum material) and sets of associated fabrication parameters.

In some embodiments, the techniques also include determining improved fabrication parameters for future fabrication of the quantum material, the improved fabrication parameters being determined at least in part on the regression model. As one non-limiting example, the determined regression models may be interpreted (e.g., using Shapley additive explanation (SHAP) values) to understand which fabrication parameters have a largest impact on the quality of the quantum material. As another non-limiting example, the improved fabrication parameters may be determined using an optimization technique (e.g., Bayesian optimization) configured to determine global minima or maxima of the figure of merit as a function of certain fabrication parameters, thereby predicting new fabrication parameter values.

In some embodiments, the quantum material may be a diamond system including color centers. For example, the color centers may be nitrogen-vacancy centers, although it should be appreciated that the color centers may be any suitable color center defect in the diamond structure, as aspects of the technology described herein are not limited to the optimization of nitrogen-vacancy center fabrication.

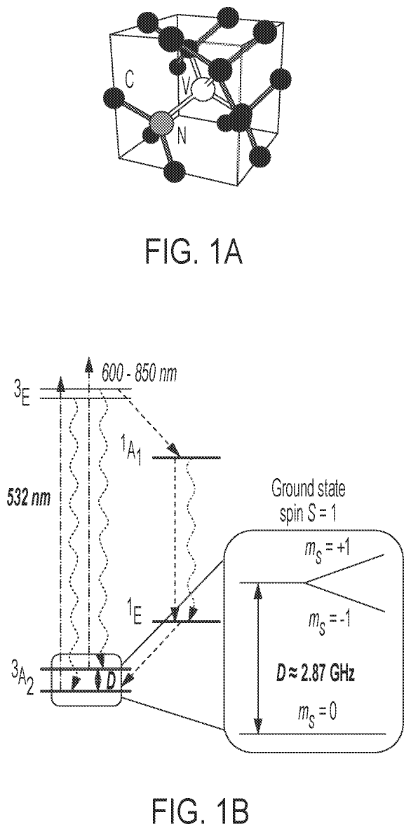

Nitrogen-vacancy (NV) centers are formed of a substitutional nitrogen atom adjacent to a lattice vacancy in the carbon crystal lattice of diamond, as depicted by FIG. 1A. FIG. 1B is a schematic diagram of an energy level structure of a negatively charged NV center (an NV⁻ center). The energy level diagram of the NV⁻ center exhibits zero-field splitting, D, between the ground state electronic spin levels mₛ=0 and mₛ=±1. The mₛ=±1 energy levels experience a Zeeman shift in the presence of an external magnetic field, an effect which forms the basis for NV magnetometry, a magnetometry technique with a high sensitivity. The NV center's spin energy levels are also sensitive to electric fields, strain, and temperature, enabling multi-modal sensing. Diamond is also chemically inert, making NV center sensors biocompatible for biological sensing applications, and the unique optical and spin-based properties of NV centers make them well-suited for implementation in quantum information platforms (e.g., quantum computing and/or quantum telecommunications platforms).

Although natural diamonds can contain NV centers and other color center defects, typically synthetic diamonds containing relevant defects are preferred for the above-described applications, as the properties of the diamond defects may be best controlled and reproduced through synthetic manufacturing techniques. Synthetic diamonds may be fabricated using multiple techniques, including by high-pressure high-temperature (HPHT) growth, chemical vapor deposition (CVD), and through the detonation of explosives. HPHT and CVD techniques can yield diamonds having a wide range of properties, and these properties can be tuned based on fabrication parameters during the HPHT and/or CVD manufacturing process. For example, the nitrogen concentration in the diamond may be varied during HPHT and/or CVD growth by varying a concentration of nitrogen gas that is present during the growth of the diamond. Isotopically pure gases (e.g., ¹²CH₄ and/or ¹⁵N₂) may be used during the diamond fabrication process to yield diamonds with better-controlled hyperfine splitting and/or reduced nuclear spin-bath contributions. Additional post-processing steps, such as irradiation, implantation, and/or annealing, can also contribute to the properties of the synthetic diamond and the NV centers contained therein.

In the example of synthetic diamond fabrication, there are at least 20 fabrication parameters related to diamond seeding, growth, post-processing, and characterization of the diamonds, resulting in a complex parameter space to navigate when attempting to fabricate synthetic diamonds with improved quantum characteristics. Additionally, these numerous parameters oftentimes have complex, non-linear correlations such that predicting the effects of changing one or more parameters may not be possible a priori and may only be discoverable after extensive experimentation.

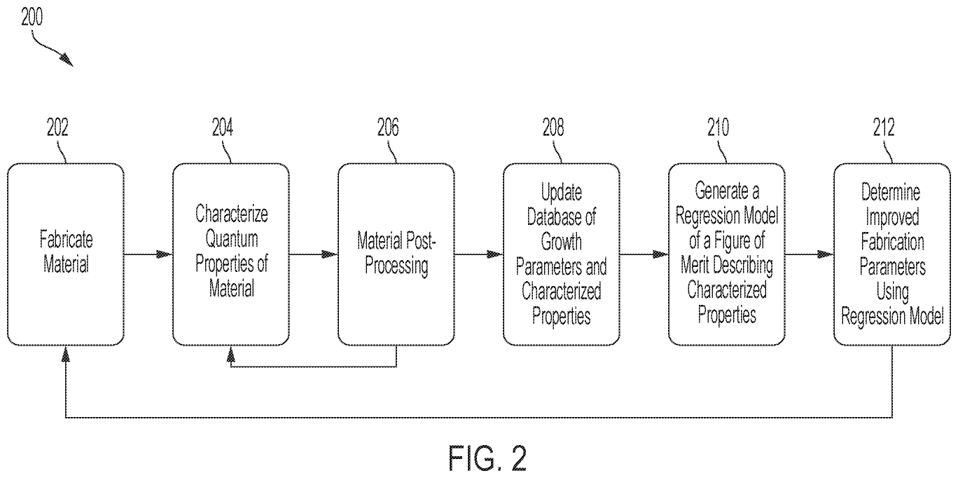

The discovery of improved fabrication parameters, and manufacture of improved quantum materials, may be accelerated using machine learning techniques. FIG. 2 is a schematic diagram of a manufacturing pipeline 200 configured to manufacture improved quantum materials, in accordance with some embodiments of the technology described herein. The pipeline 200 may begin at act 202, where a first sample of a quantum material may be fabricated using a first set of fabrication parameters. The first sample may be fabricated using one or more devices configured to fabricate the quantum material using the first set of fabrication parameters. For the example of synthetic diamonds, the one or more devices may include one or more of the non-limiting list including HPHT devices, CVD devices, etching devices, and/or defect implantation devices, although it should be appreciated that the technology described herein is not limited to these specific devices, and the quantum material may more generally be fabricated using any suitable devices (e.g., deposition devices, cleaning devices, lithography devices, etc.).

In some embodiments, after fabricating the first sample of the quantum material, the pipeline 200 may proceed to act 204, in which the quantum properties of the material may be characterized. For the example of synthetic diamonds, optical properties may be characterized. These optical properties may include characterization of relaxation times, T₁, coherence times, T₂, dephasing times,

$${T}_{2}^{*} ,$$

contrast, and/or other optical properties of the NV centers disposed in the fabricated diamond. Additionally, material properties may be characterized, including, but not limited to, the number of NV centers in the synthetic diamond, the depth of the NV centers in the diamond (e.g., how close to the surface of the diamond the NV centers are positioned), material roughness, material density, and/or other material properties of the synthetic diamond. It should be appreciated that the type of properties that are characterized may be dependent on the type of material that has been fabricated, and that the characterized properties may include additional properties not described herein, such as magnetic, electronic, phononic, temperature-dependent, or other properties, as aspects of the technology described herein are not limited in this respect.

In some embodiments, after act 204, the pipeline 200 may proceed to act 206, in which optional post-processing may be performed to further alter characteristics of the fabricated quantum material. In the case of synthetic diamonds, these post-processing activities may include, for example, irradiation to implant NV centers in the synthetic diamond and/or high temperature annealing of the synthetic diamond to cause nitrogen defects and vacancies to meet within the diamond crystal. After post-processing of the quantum material is performed, additional characterization of the quantum properties of the fabricated material may optionally be performed. The additional characterization may be the same characterization as performed prior to post-processing, or in some embodiments, the additional characterization may include characterization of different or additional properties of the fabricated material.

In some embodiments, after act 204 or after optional act 206, the pipeline 200 may proceed to act 208, in which a database of fabrication parameters and characterized quantum properties is updated based on the sample fabricated and characterized in acts 202-206. The database may be configured to store all fabrication and post-processing parameters of interest along with results of any characterization measurements. In the example of synthetic diamond, the fabrication and post-processing parameters may include one or more of the non-limiting selection of: seed miscut angle, θ, seed miscut angle, κ, deposition thickness, growth time, growth rate, seed depth (i.e., distance of the seed diamond from the plasma ball during growth), etching end temperature, total irradiation dose, lightness, [N] estimate, methane flow rate, nitrogen flow rate, pump down time, base pressure, and/or rate of rise.

In some embodiments, the database may further be configured to store a figure of merit describing the “fitness” of each sample. The figure of merit may be a measured quantity, in some embodiments, or it may be a quantity calculated based on one or more of the characterized quantum properties of the quantum material. In the case of NV centers in synthetic diamond, a figure of merit may be determined based on a particular application of the NV centers. For example, the NV centers may be planned for use in magnetometry applications, such that a figure of merit related to a magnetometry sensitivity, η, may be a suitable figure of merit for optimization. As an example, a figure of merit based on a theoretical limit of the Ramsey sensing protocol may be selected for optimization:

$$η ∼ \frac{1}{{g}_{e} {μ}_{B}} \frac{1}{C} \frac{1}{\sqrt{β {n}_{NV} - V}} \frac{\sqrt{{T}_{dead} + {T}_{2}^{*}}}{{T}_{2}^{*}} ,$$

where gₑ≈2.003 is the NV electronic g-factor, μ_{B} is the Bohr magneton, C is the measurement contrast, defined as

$$C = \frac{a - b}{a + b} ,$$

where a and b respectively denote the average number of photons detected from the mₛ=0 and mₛ=±1 states of a single NV center during a readout, β is the collection efficiency, n_{NV} is the NV density in parts-per-billion, V is the excitation volume, T_{dead} is dead time for initialization and readout, and

$${T}_{2}^{*}$$

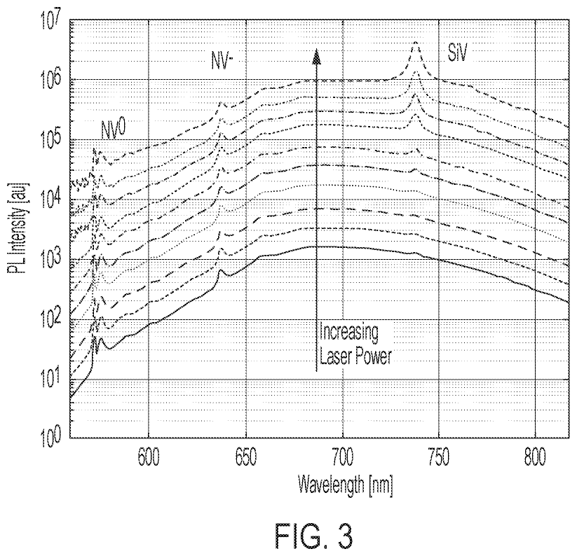

is the dephasing time. These quantities may be determined during characterization by measuring coherence times, the measurement contrast (e.g., by measuring charge state stability), and the NV signal by photoluminescence intensity. An example of measured photoluminescence spectra for increasing values of applied laser power is shown in FIG. 3; spectral deconvolution is used to isolate the relative fractions of NV⁻, NV⁰, and silicon vacancy (SiV) defects. Additional aspects of characterization of magnetometry sensitivity are described in “Sensitivity Optimization for NV-Diamond Magnetometry,” by J. F. Barry, et al., Rev. Mod. Phys. 92, 015004, published Mar. 31, 2020, which is incorporated herein by reference in its entirety.

It should be appreciated that while the specific example of a magnetometry sensitivity measure is provided herein as a figure of merit, alternative figures of merit specific to the envisioned application of the quantum material and/or specific to the class of quantum material may be used in place of the magnetometry sensitivity, as aspects of the technology described herein are not limited in this respect. For example, the figure of merit may be one or more of quantum coherence time, the concentration of defects or spins, spin linewidth, photoluminescence intensity, single photon purity, single photon indistinguishability, emission rates, or an alternate figure of merit that accounts for the quality of the chemical system.

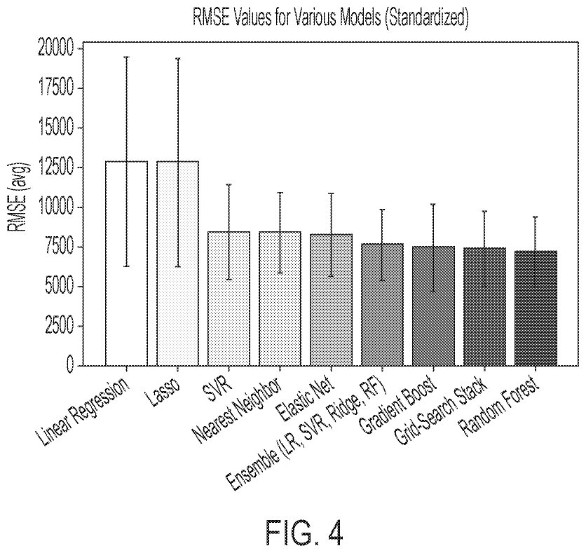

In some embodiments, after the database is updated at act 208, the pipeline 200 may proceed to act 210, in which one or more regression models of the figure of merit may be generated based on the updated database. The regression models may be generated using supervising learning techniques. For example, training data comprising related pairs of fabrication parameters and characterized quantum properties (and/or calculated or measured figures of merit) may be provided to a suitable regression model configured to generate the regression model. Examples of machine learning models that may be used to generate the regression model include, but are not limited to, random forest models, grid-search stack models, gradient boost models, elastic net models, nearest neighbor models, support vector regression (SVR) models, Lasso models, linear regression models, and/or ensemble models (e.g., including combinations of linear regression, SVR, Ridge, and/or random forest models). A regression model may be selected for further use in the pipeline 200 based on calculated values of root mean square error (RMSE), with models having lowest values of RMSE being selected for further use. FIG. 4 is a plot of calculated RMSE for different regressors used to build regression models, in accordance with some embodiments of the technology described herein.

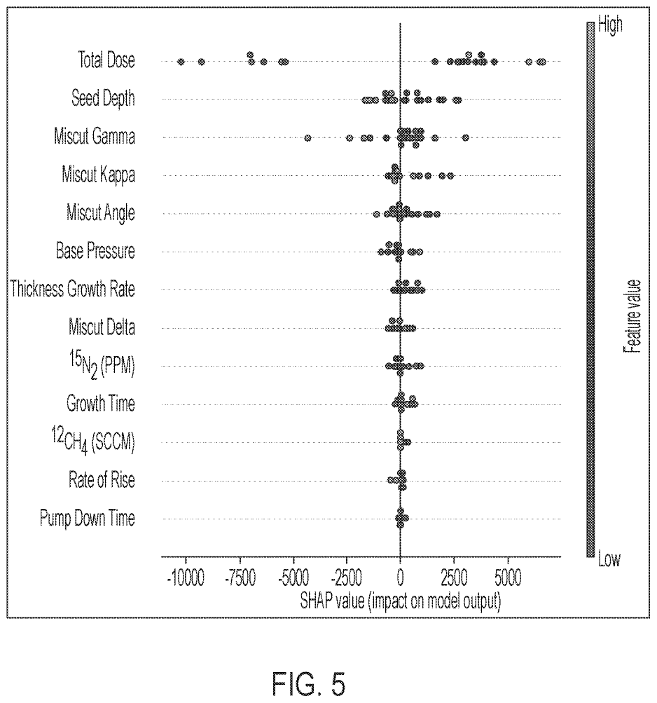

In some embodiments, after generating a regression model in act 210, the pipeline 200 may move to act 212, in which improved fabrication parameters are determined using the regression model. In some embodiments, it may be desirable to determine which fabrication parameters play the largest role in affecting the quantum material properties prior to determining new fabrication parameters. In such embodiments, the regression model generated at act 210 may be interpreted to determine the fabrication parameters having the largest effect on the quantum material properties. As one example, Shapley additive explanation (SHAP) values may be calculated using the regression model to determine the magnitude of each fabrication parameter's contribution to the regression model's predictions. Fabrication parameters associated with larger, or a largest, SHAP values may be selected as fabrication parameters that should be altered because they are most likely to improve the quantum material's final quantum properties. FIG. 5 shows illustrative SHAP values calculated for different fabrication parameters in the example of synthetic diamond systems, in accordance with some embodiments of the technology described herein.

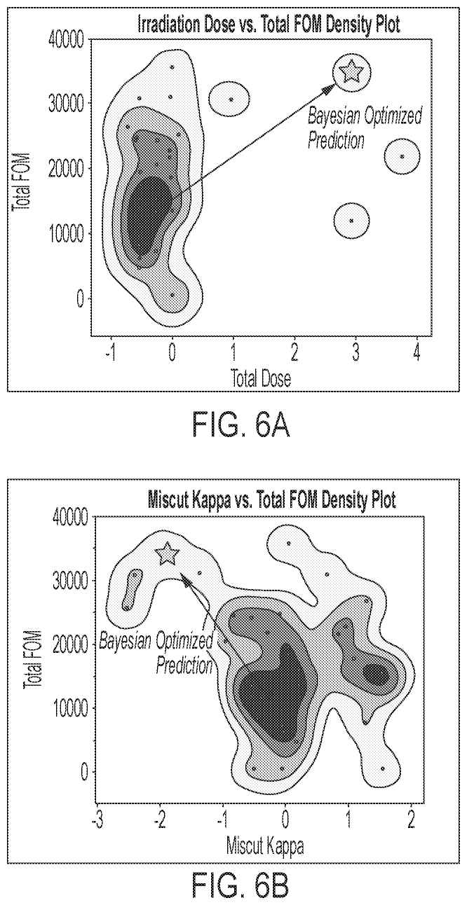

In some embodiments, improved fabrication parameters may, additionally or alternatively, be determined using optimization techniques. As one example, Bayesian optimization of the regression function may be used to predict global maxima or minima of the figure of merit as a function of one or more fabrication parameters. For example, fabrication parameters with large SHAP values may be explored using Bayesian optimization to predict new parameter spaces correlated with improved figure of merit values. FIGS. 6A and 6B are density contour plots obtained using Bayesian optimization of predicted figures of merit as a function of total irradiation dose and miscut angle, K, respectively. As shown in FIGS. 6A and 6B, Bayesian optimization can suggest new fabrication parameter values based on local maxima or minima in the multi-parameter space defined by the figure of merit and the various fabrication parameters.

Once new fabrication parameters have been determined, the pipeline 200 may return to act 202, in which a sample of the quantum material is fabricated using the new fabrication parameters. The pipeline 200 may be operated iteratively, such that new samples of the quantum material are being fabricated using improved fabrication parameters, and the database is iteratively updated such that the predictions of the machine learning models become more accurate over time. In this manner, the parameter space may be quickly and systematically explored to develop quantum materials with optimized quantum properties.

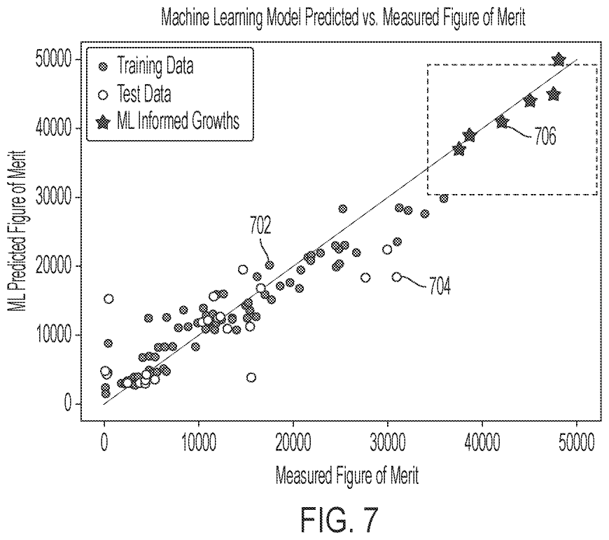

FIG. 7 is a correlation plot of machine-learning-predicted figure of merit values in comparison to measured figure of merit values. The plot includes data 702 associated with samples used to generate training data of the machine learning models, data 704 associated with samples used to test the machine learning models, and data 706 associated with samples fabricated based on fabrication parameters predicted using the machine learning techniques described herein. As seen in FIG. 7, the machine-learning-model-informed samples of data 706 have improved figures of merit compared to the training data 702 and test data 704 samples, indicating that the machine learning model techniques described herein quickly and efficiently offer combinations of growth and processing parameters that advance the quality of the quantum materials.

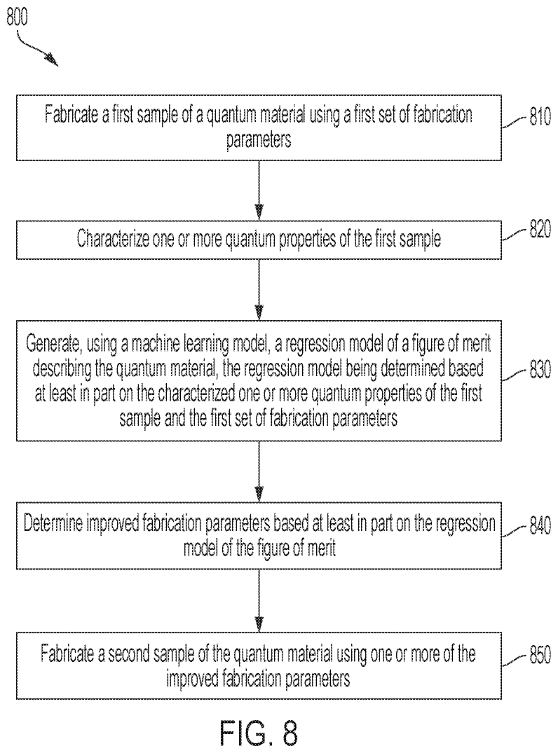

FIG. 8 is a flowchart describing a process 800 of manufacturing a quantum material, in accordance with some embodiments of the technology described herein. In some embodiments, process 800 may begin at act 810, in which a first sample of a quantum material may be fabricated using a first set of fabrication parameters. For example, the quantum material being fabricated may be a synthetic diamond including one or more color centers (e.g., NV centers and/or other suitable color centers). Alternatively or additionally, the quantum material may be any suitable chemical system that can host quantum-active defects and spins (e.g., diamond, silicon carbide, transition metal dichalcogenides, yttrium iron garnet (YIG), organic molecules, metal centered inorganic complexes, etc.), although it should be appreciated that the process 800 may be generally applicable to the manufacturing of any material having quantum properties that are to be optimized.

In some embodiments, the first sample may be fabricated using one or more devices configured to fabricate the quantum material using the first set of fabrication parameters. For the example of synthetic diamonds, the one or more devices may include one or more of the non-limiting list including HPHT devices, CVD devices, etching devices, and/or defect implantation devices, although it should be appreciated that the technology described herein is not limited to these specific devices, and the quantum material may more generally be fabricated using any suitable devices (e.g., deposition devices, cleaning devices, lithography devices, etc.).

After act 810, process 800 may proceed to act 820, in which one or more quantum properties of the first sample may be characterized. For the example of synthetic diamonds, optical properties may be characterized. These optical properties may include characterization of relaxation times, T₁, coherence times, T₂, dephasing times, T₂, contrast, and/or other optical properties of the NV centers disposed in the fabricated diamond. Additionally, material properties may be characterized, including, but not limited to, the number of NV centers in the synthetic diamond, the depth of the NV centers in the diamond (e.g., how close to the surface of the diamond the NV centers are positioned), material roughness, material density, and/or other material properties of the synthetic diamond. It should be appreciated that the type of properties that are characterized may be dependent on the type of material that has been fabricated, and that the characterized properties may include additional properties not described herein, such as magnetic, electronic, phononic, temperature-dependent, or other properties, as aspects of the technology described herein are not limited in this respect.

After act 820, process 800 may proceed to act 830, in which a regression model may be generated using a machine learning model. The regression model may be a model of a figure of merit describing the quantum material and may be determined based at least in part on the characterized one or more quantum properties of the first sample and the first set of fabrication parameters. For example, the figure of merit may be dependent on one or more quantum properties of the quantum material, and improving (e.g., maximizing and/or minimizing) the figure of merit may correlate with improving the quantum properties of the quantum material for one or more particular applications. In the example of synthetic diamonds including NV centers, the figure of merit may be based on, at least in part, one or more of measured NV center T2* dephasing times, contrast, and/or fluorescence.

In some embodiments, the regression models may be generated using supervising learning techniques. For example, the machine learning model may be learned using pairs of calculated figures of merit (e.g., calculated based on one or more measured quantum properties of characterized samples of the quantum material) and sets of fabrication parameters used to fabricate associated samples of the quantum material. In the example of synthetic diamonds including NV or color centers, the fabrication parameters may include one or more of seed miscut angles, growth time, seed depth, total irradiation dose, methane flow rate, nitrogen flow rate, pump down times, base pressure, and/or rate of rise. Examples of machine learning models that may be used to generate the regression model include, but are not limited to, random forest models, grid-search stack models, gradient boost models, elastic net models, nearest neighbor models, support vector regression (SVR) models, Lasso models, linear regression models, and/or ensemble models (e.g., including combinations of linear regression, SVR, Ridge, and/or random forest models).

After act 830, process 800 may proceed to act 840, in which improved fabrication parameters may be determined based at least in part on the generated regression model of the figure of merit. In some embodiments, determining the improved fabrication parameters may include determining which fabrication parameters play the largest role in affecting the quantum material properties. In such embodiments, the generated regression model may be quantitatively interpreted to determine the fabrication parameters having the largest effect on the quantum material properties. As one example, SHAP values may be calculated using the regression model to determine the magnitude of each fabrication parameter's contribution to the regression model's predictions. Fabrication parameters associated with larger, or a largest, SHAP values may be selected as fabrication parameters that should be altered when generating the improved fabrication parameters because the fabrication parameters associated with larger SHAP values are most likely to improve the quantum material's final quantum properties.

In some embodiments, improved fabrication parameters may, additionally or alternatively, be determined using optimization techniques. As one example, Bayesian optimization of the regression function may be used to predict global maxima or minima of the figure of merit as a function of one or more fabrication parameters. For example, fabrication parameters with large SHAP values may be explored using Bayesian optimization to predict new parameter spaces correlated with improved figure of merit values. Alternatively or additionally, one or more fabrication parameters, regardless of SHAP values, may be explored using Bayesian optimization to generate the improved fabrication parameters.

Acts 830 and/or 840 may be executed using any suitable computing device. For example, in some embodiments, acts 830 and/or 840 may be performed by a computing device co-located (e.g., in the same room) with a fabrication device used to fabricate the first sample of the quantum material. As another example, in some embodiments, acts 830 and/or 840 may be performed by one or more processors located on one of the fabrication devices used to fabricate the first sample of the quantum material. Alternately, in some embodiments, acts 830 and/or 840 may be performed by one or more processors located remotely from the devices used to fabricate the first sample of the quantum material (e.g., as part of a cloud computing environment).

After act 840, process 800 may proceed to act 850, in which a second sample of the quantum material may be fabricated using one or more of the improved fabrication parameters, yielding a second sample of the quantum material having improved quantum properties relative to the first sample of the quantum material. It should be appreciated that acts 810-850 may be iteratively performed one or more times to improve the regression models used to predict optimized fabrication parameters and to improve the quantum properties of the quantum material.

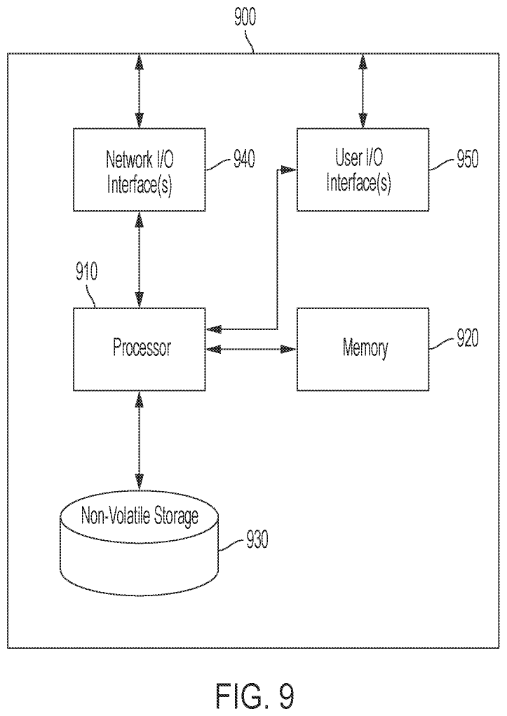

An illustrative implementation of a computer system 900 that may be used in connection with any of the embodiments of the technology described herein (e.g., such as the methods of FIG. 2 and/or FIG. 8) is shown in FIG. 9. The computer system 900 includes one or more processors 910 and one or more articles of manufacture that comprise non-transitory computer-readable storage media (e.g., memory 920 and one or more non-volatile storage media 930). The processor 910 may control writing data to and reading data from the memory 920 and the non-volatile storage device 930 in any suitable manner, as the aspects of the technology described herein are not limited to any particular techniques for writing or reading data. To perform any of the functionality described herein, the processor 910 may execute one or more processor-executable instructions stored in one or more non-transitory computer-readable storage media (e.g., the memory 920), which may serve as non-transitory computer-readable storage media storing processor-executable instructions for execution by the processor 910.

Computing device 900 may also include a network input/output (I/O) interface 940 via which the computing device may communicate with other computing devices (e.g., over a network), and may also include one or more user I/O interfaces 950, via which the computing device may provide output to and receive input from a user. The user I/O interfaces 950 may include devices such as a keyboard, a mouse, a microphone, a display device (e.g., a monitor or touch screen), speakers, a camera, and/or various other types of I/O devices.

The above-described embodiments can be implemented in any of numerous ways. For example, the embodiments may be implemented using hardware, software, or a combination thereof. When implemented in software, the software code can be executed on any suitable processor (e.g., a microprocessor) or collection of processors, whether provided in a single computing device or distributed among multiple computing devices. It should be appreciated that any component or collection of components that perform the functions described above can be generically considered as one or more controllers that control the above-discussed functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments described herein comprises at least one computer-readable storage medium (e.g., RAM, ROM, EEPROM, flash memory or other memory technology, CD-ROM, digital versatile disks (DVD) or other optical disk storage, magnetic cassettes, magnetic tape, magnetic disk storage or other magnetic storage devices, or other tangible, non-transitory computer-readable storage medium) encoded with a computer program (i.e., a plurality of executable instructions) that, when executed on one or more processors, performs the above-discussed functions of one or more embodiments. The computer-readable medium may be transportable such that the program stored thereon can be loaded onto any computing device to implement aspects of the techniques discussed herein. In addition, it should be appreciated that the reference to a computer program which, when executed, performs any of the above-discussed functions, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques discussed herein.

The foregoing description of implementations provides illustration and description but is not intended to be exhaustive or to limit the implementations to the precise form disclosed. Modifications and variations are possible in light of the above teachings or may be acquired from practice of the implementations. In other implementations the methods depicted in these figures may include fewer operations, different operations, differently ordered operations, and/or additional operations. Further, non-dependent blocks may be performed in parallel.

It will be apparent that example aspects, as described above, may be implemented in many different forms of software, firmware, and hardware in the implementations illustrated in the figures. Further, certain portions of the implementations may be implemented as a “module” that performs one or more functions. This module may include hardware, such as a processor, an application-specific integrated circuit (ASIC), or a field-programmable gate array (FPGA), or a combination of hardware and software.

Various aspects of the embodiments described above may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Having thus described several aspects and embodiments of the technology set forth in the disclosure, it is to be appreciated that various alterations, modifications, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize or be able to ascertain using no more than routine experimentation many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

The above-described embodiments can be implemented in any of numerous ways. One or more aspects and embodiments of the present disclosure involving the performance of processes or methods may utilize program instructions executable by a device (e.g., a computer, a processor, or other device) to perform, or control performance of, the processes or methods. In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above. The computer readable medium or media can be transportable, such that the program or programs stored thereon can be loaded onto one or more different computers or other processors to implement various ones of the aspects described above. In some embodiments, computer readable media may be non-transitory media.

The terms “program” or “software” are used herein in a generic sense to refer to any type of computer code or set of computer-executable instructions that can be employed to program a computer or other processor to implement various aspects as described above. Additionally, it should be appreciated that according to one aspect, one or more computer programs that when executed perform methods of the present disclosure need not reside on a single computer or processor but may be distributed in a modular fashion among a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

When implemented in software, the software code can be executed on any suitable processor or collection of processors, whether provided in a single computer or distributed among multiple computers.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone, a tablet, or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The use of “coupled” or “connected” is meant to refer to elements, or signals, that are either directly linked to one another or are linked through intermediate components. Elements that are not “coupled” or “connected” are “decoupled” or “disconnected.”

The use of “between” in a coupled signal chain is not meant to require a particular direction of signal flow in the signal chain unless stated otherwise. For instance, where element B is described as coupled between elements A and C in a signal chain, signals may flow from element A to element C through element B and/or from element C to element A through element B unless stated otherwise.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B,” when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

The terms “approximately” and “about” may be used to mean within ±20% of a target value in some embodiments, within ±10% of a target value in some embodiments, within ±5% of a target value in some embodiments, within ±2% of a target value in some embodiments. The terms “approximately” and “about” may include the target value.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

1. A method of manufacturing a quantum material, the method comprising:
fabricating a first sample of the quantum material using a first set of fabrication parameters;
characterizing one or more quantum properties of the first sample;
generating, using a machine learning model, a regression model of a figure of merit describing the quantum material, the regression model being determined based at least in part on the characterized one or more quantum properties of the first sample and the first set of fabrication parameters;
determining improved fabrication parameters based at least in part on the regression model of the figure of merit; and
fabricating a second sample of the quantum material using one or more of the improved fabrication parameters.

2. The method of claim 1, wherein fabricating the first sample and fabricating the second sample comprises fabricating diamond samples, the diamond samples including one or more color centers.

3. The method of claim 2, wherein fabricating the diamond samples including one or more color centers comprises fabricating diamond samples including one or more nitrogen-vacancy centers.

4. The method of claim 1, wherein generating the regression model comprises performing gradient boost regression, random forest regression, and/or stacking regression.

5. The method of claim 1, wherein determining the improved fabrication parameters comprises calculating SHAP values associated with fabrication parameters of the first set of fabrication parameters and predicted effects of the fabrication parameters on the figure of merit.

6. The method of claim 5, wherein determining the improved fabrication parameters comprises determining one of the first set of fabrication parameters having a largest impact on the figure of merit based at least in part on the calculated SHAP values.

7. The method of claim 1, wherein determining the improved fabrication parameters comprises determining the improved fabrication parameters using Bayesian optimization.

8. The method of claim 7, wherein using Bayesian optimization comprises determining local minima and/or maxima of the figure of merit as a function of one fabrication parameter of the first set of fabrication parameters to determine one of the improved fabrication parameters.

9. The method of claim 1, wherein generating the regression model based at least in part on the characterized one or more quantum properties of the first sample comprises generating the regression model based at least in part on one or more of measured T2* dephasing times, contrast, and/or fluorescence.

10. The method of claim 1, wherein generating the regression model based at least in part on the first set of fabrication parameters comprises generating the regression model based at least in part on one or more of seed miscut angles, growth time, seed depth, total irradiation dose, methane flow rate, nitrogen flow rate, pump down times, base pressure, and/or rate of rise.

11. A system, comprising:
one or more devices configured to fabricate a first sample and a second sample of a quantum material;
at least one computer processor; and
at least one non-transitory computer readable medium storing instructions that, when executed by the at least one computer processor, cause the at least one computer processor to perform a method of improving manufacture of a quantum material, the method comprising:
generating, using a machine learning model, a regression model of a figure of merit describing the quantum material, the regression model being determined based at least in part on characterized one or more quantum properties of the first sample and a first set of fabrication parameters used by the one or more devices to fabricate the first sample; and
determining improved fabrication parameters based at least in part on the regression model of the figure of merit, the determined improved fabrication parameters to be used by the one or more devices to fabricate the second sample.

12. The system of claim 11, wherein the one or more devices are configured to fabricate diamond samples including one or more color centers.

13. The system of claim 12, wherein the one or more devices are configured to fabricate diamond samples including one or more nitrogen-vacancy centers.

14. The system of claim 11, wherein generating the regression model comprises performing gradient boost regression, random forest regression, and/or stacking regression.

15. The system of claim 11, wherein determining the improved fabrication parameters comprises calculating SHAP values associated with fabrication parameters of the first set of fabrication parameters and predicted effects of the fabrication parameters on the figure of merit.

16. The system of claim 15, wherein determining the improved fabrication parameters comprises determining one of the first set of fabrication parameters having a largest impact on the figure of merit based at least in part on the calculated SHAP values.

17. The system of claim 11, wherein determining the improved fabrication parameters comprises determining the improved fabrication parameters using Bayesian optimization.

18. The system of claim 17, wherein using Bayesian optimization comprises determining local minima and/or maxima of the figure of merit as a function of one fabrication parameter of the first set of fabrication parameters to determine one of the improved fabrication parameters.

19. The system of claim 11, wherein generating the regression model based at least in part on the characterized one or more quantum properties of the first sample comprises generating the regression model based at least in part on one or more of measured T2* dephasing times, contrast, and/or fluorescence.

20. The system of claim 11, wherein generating the regression model based at least in part on the first set of fabrication parameters comprises generating the regression model based at least in part on one or more of seed miscut angles, growth time, seed depth, total irradiation dose, methane flow rate, nitrogen flow rate, pump down times, base pressure, and/or rate of rise.

21. (canceled)
